(19) United States
(12) Patent Application Publication
(10) Pub. No.: US 2026/0209647 A1
(43) Pub. Date: Jul. 23, 2026

(54) COMPOSITION, METHOD OF TREATING METAL-CONTAINING FILM BY USING THE COMPOSITION, AND METHOD OF MANUFACTURING ELECTRONIC DEVICE BY USING THE COMPOSITION

(71) Applicant: Samsung Electronics Co., Ltd., Suwon-si (KR)

(72) Inventors: Wonsik YOON, Suwon-si (KR); Sungmin KIM, Suwon-si (KR); Youngchan KIM, Suwon-si (KR); Jina KIM, Suwon-si (JP); Jinhye BAE, Suwon-si (KR); Minhyung CHO, Suwon-si (KR); Donghoon HA, Suwon-si (KR); Cheol HAM, Suwon-si (KR); Kyuyoung HWANG, Suwon-si (KR)

(73) Assignee: Samsung Electronics Co., Ltd., Suwon-si (KR)

(21) Appl. No.: 19/436,783

(22) Filed: Dec. 30, 2025

(30) Foreign Application Priority Data
Jan. 17, 2025 (KR) 10-2025-0007549

(51) Int. Cl. C11D 7/32; C11D 7/16

(52) U.S. Cl. CPC C11D 7/3209; C11D 7/16; C11D 2111/22

(57) ABSTRACT

Provided are a composition, a method of treating a metal-containing film by using the composition, and a method of manufacturing an electronic device by using the composition. The composition may include an oxidizing agent, an ammonium-based buffer, and an etching controller. The etching controller may include a compound represented by Formula 1.

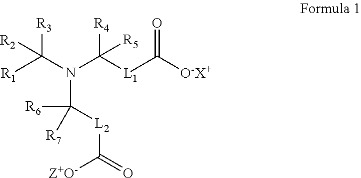

A description of Formula 1 is provided in the present specification.

### CROSS-REFERENCE TO RELATED APPLICATIONS

This application is based on and claims priority under 35 U.S.C. § 119 to Korean Patent Application Nos. 10-2025-0007549, filed on Jan. 17, 2025, in the Korean Intellectual Property Office, the disclosure of which is incorporated by reference herein in its entirety.

### BACKGROUND

### 1. Field

The disclosure relates to a composition, a method of treating a metal-containing film by using the composition, and a method of manufacturing an electronic device by using the composition.

### 2. Description of the Related Art

To satisfy excellent performance and low prices demanded by consumers, electronic devices, such as semiconductor devices, increased integration and improved reliability may be advantageous. As the degree of integration of the semiconductor devices increases, damage to components of the semiconductor devices during a process of manufacturing the semiconductor devices may further affects the reliability and/or electrical characteristics of semiconductor devices. In particular, in the process of manufacturing the semiconductor devices, various processing processes such as etching, cleaning, and the like may be performed on a film (e.g., a metal-containing film). To more effectively perform a treatment process on a metal-containing film, compositions having an appropriate etching rate and/or excellent cleaning ability may be advantageous.

### SUMMARY

Provided are a composition having improved etching selectivity and/or cleaning performance, a method of treating a metal-containing film by using the composition, and/or a method of manufacturing an electronic device by using the composition.

Additional aspects will be set forth in part in the description which follows and, in part, will be apparent from the description, or may be learned by practice of the presented embodiments of the disclosure.

According to an embodiment of the disclosure, a composition (e.g., an etching composition) may include:
an oxidizing agent, an ammonium-based buffer, and an etching controller,
wherein the etching controller may include a compound represented by Formula 1:

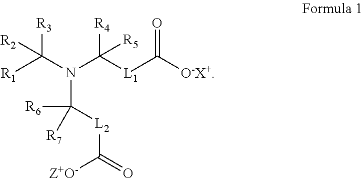

In Formula 1,
R₁ may be a C₁-C₃₀ alkyl group or a C₂-C₃₀ alkenyl group,
R₂ to R₇ may each independently be hydrogen, a C₁-C₃₀ alkyl group, or a C₂-C₃₀ alkenyl group,
L₁ and L₂ may each independently be a C₁-C₃₀ alkylene group or a C₂-C₃₀ alkenylene group,
X and Z may each independently be hydrogen, an alkali metal, or an ammonium group,
at least one methylene group included in one or more of R₁ to R₇ may optionally be substituted with O or S, and
at least one hydrogen included in one or more of R₁ to R₇, L₁, and L₂ may optionally be substituted with a halogen atom, a hydroxyl group, a thiol group, a C₁-C₃₀ alkoxy group, or a C₁-C₃₀ alkylthio group.

According to an embodiment of the disclosure, a method of treating (e.g., etching) a metal-containing film may include
preparing a substrate on which a metal-containing film is provided; and
contacting the metal-containing film with the composition.

A metal included in the metal-containing film may include titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or any combination thereof.

In some embodiments, by the contacting of the metal-containing film with the composition, at least a portion of the metal-containing film may be at least one of etched or cleaned.

In some embodiments, the metal-containing film may have a first region and a second region, and
a second etching rate of the composition for etching the second region may be greater than a first etching rate of the composition for etching the first region.

In some embodiments, the first region may include Co, Cu, or a combination thereof, and the second region may include titanium nitride.

In some embodiments, by the contacting of the metal-containing film with the composition, at least a portion of the metal-containing film may be cleaned by removing residues on the surface of the metal-containing film, and
the residue may include an etching gas residue, a polymer residue, a metal-containing residue, or any combination thereof.

According to an embodiment of the disclosure, a method of manufacturing an electronic device (e.g., semiconductor device) may include
preparing a substrate on which a metal-containing film is provided,
contacting the metal-containing film with the composition, and
manufacturing an electronic device by performing one or more subsequent manufacturing process(es).

It will be understood that the technical features described herein in connection with the method of treating the metal-containing film are also applicable to the method of manufacturing the electronic device (e.g., semiconductor device).

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a process flow chart of an embodiment of a method of manufacturing an electronic device;

FIGS. 2 and 3 are each a diagram briefly explaining an embodiment of a method of treating a metal-containing film; and

FIGS. 4A to 4J are each a cross-sectional view describing an embodiment of a trench-via hole patterning process for bitline electrode formation.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figure, to explain aspects. As used herein, the term “and/or” includes any and all combinations of one or more of the associated listed items. Expressions such as “at least one of,” when preceding a list of elements, modify the entire list of elements and do not modify the individual elements of the list. For example, “at least one of A, B, and C,” and similar language (e.g., “at least one selected from the group consisting of A, B, and C” and “at least one of A, B, or C”) may be construed as A only, B only, C only, or any combination of two or more of A, B, and C, such as, for instance, ABC, AB, BC, and AC.

When the terms “about” or “substantially” are used in this specification in connection with a numerical value, it is intended that the associated numerical value includes a manufacturing or operational tolerance (e.g., +10%) around the stated numerical value. Moreover, when the words “generally” and “substantially” are used in connection with geometric shapes, it is intended that precision of the geometric shape is not required but that latitude for the shape is within the scope of the disclosure. Further, regardless of whether numerical values or shapes are modified with “about” or “substantially,” it will be understood that these values and shapes should be construed as including a manufacturing or operational tolerance (e.g., +10%) around the stated numerical values or shapes. When ranges are specified, the range includes all values therebetween such as at increments of 0.1%.

### Metal-Containing Film

A metal included in an metal-containing film may be an alkali metal (e.g., sodium (Na), potassium (K), rubidium (Rb), cesium (Cs), etc.), an alkaline earth metal (e.g., beryllium (Be), magnesium (Mg), calcium (Ca), strontium (Sr), barium (Ba), etc.), a lanthanide metal (e.g., lanthanum (La), europium (Eu), terbium (Tb), ytterbium (Yb), etc.), a transition metal (e.g., scandium (Sc), yttrium (Y), titanium (Ti), zirconium (Zr), hafnium (Hf), vanadium (V), niobium (Nb), tantalum (Ta), chromium (Cr), molybdenum (Mo), tungsten (W), manganese (Mn), iron (Fe), ruthenium (Ru), osmium (Os), cobalt (Co), rhodium (Rh), nickel (Ni), copper (Cu), silver (Ag), zinc (Zn), etc.), a post-transition metal (e.g., aluminum (Al), gallium (Ga), indium (In), thallium (Tl), tin (Sn), bismuth (Bi), etc.), or any combination thereof.

In an embodiment, a metal included in the metal-containing film may include Ti, In, Al, Co, La, Sc, Ga, W, Mo, Ru, Zn, Hf, Cu, or any combination thereof.

In one or more embodiments, the metal-containing film may include two or more different types of metals.

In one or more embodiments, the metal included in the metal-containing film may include
i) Ti, and
ii) In, Al, Co, La, Sc, Ga, W, Mo, Ru, Zn, Hf, Cu, or any combination thereof.

In one or more embodiments, the metal-containing film may include Al, Ti, La, Co, Cu, or any combination thereof.

In one or more embodiments, the metal-containing film may include Ti.

In one or more embodiments, the metal-containing film may include Co.

In one or more embodiments, the metal-containing film may include Cu.

In one or more embodiments, the metal-containing film may include Ti and Co.

In one or more embodiments, the metal-containing film may include Ti and Cu.

The metal-containing film may include a metal, metal nitride, metal oxide, metal oxynitride, or a combination thereof.

In an embodiment, the metal-containing film may include a metal, metal nitride, metal oxide, metal oxynitride, or any combination thereof, and each of the aforementioned metal, a metal included in the metal nitride, a metal included in the metal oxide, and a metal included in the metal oxynitride may include Ti, In, Al, Co, La, Sc, Ga, W, Mo, Ru, Zn, Hf, Cu, or any combination thereof.

In one or more embodiments, the metal-containing film may include the metal nitride.

In one or more embodiments, the metal-containing film may include the aforementioned metal (e.g., Co, Cu, or a combination thereof).

In one or more embodiments, the metal-containing film may include metal nitride and a metal (e.g., Co, Cu, or a combination thereof). For example, a metal included in the metal nitride may be different from the metal.

In one or more embodiments, the metal-containing film may include metal nitride and a metal (e.g., Co, Cu, or a combination thereof), and a metal included in the metal nitride may include In, Ti, Al, La, Sc, Ga, Zn, Hf, or any combination thereof.

In one or more embodiments, the metal-containing film may include titanium nitride and a metal (e.g., Co, Cu, or a combination thereof), and the titanium nitride may optionally further include In, Al, La, Sc, Ga, Hf, Zn, W, Si, or any combination thereof.

In one or more embodiments, the metal-containing film may include titanium nitride, titanium nitride further including Al (e.g., titanium/aluminum nitride or TiAlN), titanium nitride further including La, or the like.

In one or more embodiments, the metal-containing film may include metal oxide. A metal included in the metal oxide may include Ti, Al, La, Sc, Ga, Hf, or any combination thereof. In an embodiment, the metal-containing film may include aluminum oxide (e.g., Al₂O₃), indium gallium zinc oxide (IGZO), or the like.

In one or more embodiments, the metal-containing film may include the metal nitride and the metal oxide.

In one or more embodiments, the metal-containing film may further include, in addition to the metal, a metalloid (e.g., boron (B), silicon (Si), germanium (Ge), arsenic (As), antimony (Sb), tellurium (Te), etc.), a non-metal (e.g., nitrogen (N), phosphorus (P), oxygen (O), sulfur(S), selenium (Se), etc.), or any combination thereof.

For example, the metal-containing film may further include silicon oxide.

In an embodiment, the metal-containing film may include
a) i) titanium nitride or ii) titanium nitride further including In, Al, La, Sc, Ga, Hf, Zn, W, Si, or any combination thereof, and
b) Co, Cu, or a combination thereof.

The metal-containing film may have a single-layer structure consisting of one or more materials or a multi-layer structure including different materials. A plurality of films included in the multi-layer structure may be vertically stacked or horizontally arranged. The single-layer structure and the multi-layer structure may have various three-dimensional patterns (e.g., via holes, trenches, etc.).

In an embodiment, the metal-containing film may have a first region and a second region, and a second etching rate of the composition for etching the second region may be greater than a first etching rate of the composition for etching the first region. During a treatment process (e.g., an etching process, a cleaning process, etc.) on the metal-containing film, at least a portion of the first region and at least a portion of the second region may be in contact with the composition, and since the second etching rate is greater than the first etching rate, the second region may be etched faster than the first region. The first region and the second region may be spaced apart from each other.

For example, the first region may include a metal, metal oxide (e.g., aluminum oxide), silicon oxide, or a combination thereof.

In an embodiment, the first region may include Co, Cu, or a combination thereof.

In one or more embodiments, the second region may include metal nitride (e.g., titanium nitride).

In one or more embodiments, the second region may include i) titanium nitride, ii) titanium nitride further including In, Al, La, Sc, Ga, Zn, Hf, or any combination thereof (e.g., TiAlN), or iii) a combination thereof.

In one or more embodiments, each of the first region and the second region may include i) titanium nitride, ii) titanium nitride further including In, Al, La, Sc, Ga, Zn, Hf, or any combination thereof, or iii) a combination thereof.

In one or more embodiment, the first region may include Co, Cu, or a combination thereof, whereas the second region may not include Co and Cu.

In one or more embodiments, the first region may include Co, Cu, or a combination thereof, and the second region may include i) titanium nitride, ii) titanium nitride further including In, Al, La, Sc, Ga, Zn, Hf, or any combination thereof (e.g., TiAlN), or iii) a combination thereof.

In one or more embodiments, the first region may include Co, Cu, or a combination thereof, and the second region may include titanium nitride, titanium nitride further including Al (e.g., TiAlN), or any combination thereof.

In one or more embodiments, the first region may include a Co film, a Cu film, or a combination thereof, and the second region may include a titanium nitride film, a titanium nitride film further including Al (e.g., a titanium/aluminum nitride film or a TiAlN film), or any combination thereof.

In one or more embodiments, the first region may include a Co film, a Cu film, or a combination thereof, and the second region may include a titanium nitride film or a titanium nitride film further including Al (e.g., a titanium/aluminum nitride film or a TiAlN film).

Etching a film described herein may refer to removing at least some of materials constituting a film to be etched.

### Composition

The composition may include an oxidizing agent, an ammonium-based buffer, and an etching controller.

The composition may be used in various treatment processes, such as an etching process, a cleaning process, a polishing process, etc., for the metal-containing film.

The composition may further include water. The composition may further include a chelating agent (e.g., ethylenediaminetetraacetic acid (EDTA)).

### Oxidizing Agent

The oxidizing agent may serve to etch at least a portion of the metal-containing film by oxidizing at least some of metals included in the metal-containing film and forming a water-soluble complex, and may include, for example, at least one of hydrogen peroxide, nitric acid, and ammonium sulphate.

In an embodiment, the oxidizing agent may include hydrogen peroxide.

In one or more embodiments, the oxidizing agent is hydrogen peroxide.

An amount (weight) of the oxidizing agent may be, for example, per 100 wt % of the composition, in a range of about 16 wt % to about 50 wt %, about 18 wt % to about 50 wt %, about 20 wt % to about 50 wt %, about 22 wt % to about 50 wt %, about 25 wt % to about 50 wt %, about 16 wt % to about 45 wt %, about 18 wt % to about 45 wt %, about 20 wt % to about 45 wt %, about 22 wt % to about 45 wt %, about 25 wt % to about 45 wt %, about 16 wt % to about 40 wt %, about 18 wt % to about 40 wt %, about 20 wt % to about 40 wt %, about 22 wt % to about 40 wt %, about 25 wt % to about 40 wt %, about 16 wt % to about 35 wt %, about 18 wt % to about 35 wt %, about 20 wt % to about 35 wt %, about 22 wt % to about 35 wt %, about 25 wt % to about 35 wt %, about 16 wt % to about 30 wt %, about 18 wt % to about 30 wt %, about 20 wt % to about 30 wt %, about 22 wt % to about 30 wt %, about 25 wt % to about 30 wt %, about 16 wt % to about 27 wt %, about 18 wt % to about 27 wt %, about 20 wt % to about 27 wt %, about 22 wt % to about 27 wt %, or about 25 wt % to about 27 wt %.

When the amount of the oxidizing agent within the ranges above is satisfied, the composition may have both excellent etching selectivity and/or excellent cleaning performance at the same time.

### Ammonium-Based Buffer

The ammonium-based buffer may serve to maintain a high concentration of anions generated by the oxidizing agent and to stabilize a water-soluble composite generated when the anions oxidize at least a portion of a metal included in the metal-containing film. By using such an ammonium-based buffer, at least a portion of the metal-containing film may be effectively etched.

The ammonium-based buffer may include an ammonium group.

In an embodiment, the ammonium-based buffer may include an ammonium group represented by N (A₁₁)(A₁₂)(A₁₃)(A₁₄), wherein A₁₁ to A₁₄ may each independently be hydrogen, a C₁-C₃₀ alkyl group, a C₂-C₃₀ alkenyl group, a C₃-C₃₀ carbocyclic group, or a C₁-C₃₀ heterocyclic group.

For example, A₁₁ to A₁₄ may each independently be hydrogen or a C₁-C₁₀ alkyl group.

In one or more embodiments, the ammonium-based buffer may include hydroxide, acetate, bicarbonate, benzoate, carbonate, formate, nitrate, hydrogensulfate, carbamate, sulfamate, citrate, phosphate, sulfite, sulfobenzoate, oxalate, lactate, tartrate, dihydrogencitrate, glutamate, salicylate, bioxalate, octanoate, propionate, glycolate, gluconate, or any combination thereof.

In one or more embodiments, the ammonium-based buffer may include phosphate or hydroxide. The phosphate as the ammonium-based buffer refers to a phosphate-containing ammonium-based compound.

In one or more embodiments, the ammonium-based buffer may include a phosphate-containing ammonium-based compound (e.g., a compound represented by Formula 11-1, a compound represented by Formula 11-2, a compound represented by Formula 11-3, or any combination thereof), a hydroxide-containing ammonium-based compound (e.g., a compound represented by Formula 11-4), or a combination thereof.

In one or more embodiments, the ammonium-based buffer may include the phosphate-containing ammonium-based compound.

In one or more embodiments, the ammonium-based buffer may include a compound represented by Formula 11-1, a compound represented by Formula 11-2, a compound represented by Formula 11-3, a compound represented by Formula 11-4, or any combination thereof:

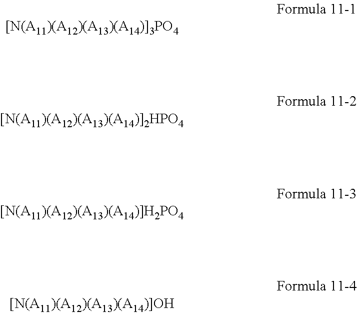

wherein a description of each of Formulae 11-1 to 11-4 may each be as defined herein.

In one or more embodiments, the ammonium-based buffer may include ammonium phosphate ((NH₄)₃PO₄), diammonium monohydrogen phosphate ((NH₄)₂HPO₄), ammonium dihydrogen phosphate ((NH₄)H₂PO₄), tris(tetramethylammonium) phosphate ([N(CH₃)₄]₃PO₄), bis(tetramethylammonium) monohydrogen phosphate ([N(CH₃)₄]₂HPO₄), tetramethylammonium dihydrogen phosphate ([N(CH₃)₄]H₂PO₄), ammonium hydroxide, tetramethylammonium hydroxide (TMAH), or any combination thereof.

An amount (weight) of the ammonium-based buffer may be, for example, per 100 wt % of the composition, in a range of about 0.01 wt % to about 10 wt %, about 0.05 wt % to about 10 wt %, about 0.1 wt % to about 10 wt %, about 0.3 wt % to about 10 wt %, about 0.5 wt % to about 10 wt %, about 0.01 wt % to about 7 wt %, about 0.05 wt % to about 7 wt %, about 0.1 wt % to about 7 wt %, about 0.3 wt % to about 7 wt %, about 0.5 wt % to about 7 wt %, about 0.01 wt % to about 4 wt %, about 0.05 wt % to about 4 wt %, about 0.1 wt % to about 4 wt %, about 0.3 wt % to about 4 wt %, about 0.5 wt % to about 4 wt %, about 0.01 wt % to about 2 wt %, about 0.05 wt % to about 2 wt %, about 0.1 wt % to about 2 wt %, about 0.3 wt % to about 2 wt %, about 0.5 wt % to about 2 wt %, about 0.01 wt % to about 1 wt %, about 0.05 wt % to about 1 wt %, about 0.1 wt % to about 1 wt %, about 0.3 wt % to about 1 wt %, or about 0.5 wt % to about 1 wt %.

In an embodiment, the ammonium-based buffer may include the phosphate-containing ammonium-based compound, the hydroxide-containing ammonium-based compound, or a combination thereof, and an amount (weight) of the phosphate-containing ammonium-based compound and an amount (weight) of the hydroxide-containing ammonium-based compound may each independently be, for example, per 100 wt % of the composition, in a range of about 0.01 wt % to about 10 wt %, about 0.05 wt % to about 10 wt %, about 0.1 wt % to about 10 wt %, about 0.3 wt % to about 10 wt %, about 0.5 wt % to about 10 wt %, about 0.01 wt % to about 7 wt %, about 0.05 wt % to about 7 wt %, about 0.1 wt % to about 7 wt %, about 0.3 wt % to about 7 wt %, about 0.5 wt % to about 7 wt %, about 0.01 wt % to about 4 wt %, about 0.05 wt % to about 4 wt %, about 0.1 wt % to about 4 wt %, about 0.3 wt % to about 4 wt %, about 0.5 wt % to about 4 wt %, about 0.01 wt % to about 2 wt %, about 0.05 wt % to about 2 wt %, about 0.1 wt % to about 2 wt %, about 0.3 wt % to about 2 wt %, about 0.5 wt % to about 2 wt %, about 0.01 wt % to about 1 wt %, about 0.05 wt % to about 1 wt %, about 0.1 wt % to about 1 wt %, about 0.3 wt % to about 1 wt %, about 0.5 wt % to about 1 wt %, about 0.01 wt % to about 0.7 wt %, about 0.05 wt % to about 0.7 wt %, about 0.1 wt % to about 0.7 wt %, about 0.3 wt % to about 0.7 wt %, about 0.5 wt % to about 0.7 wt %, or about 0.3 wt % to about 0.5 wt %.

When the amount of the ammonium-based buffer within the ranges above is satisfied, the composition may have both improved (and/or excellent) etching selectivity and improved (and/or excellent) cleaning performance at the same time.

### Etching Controller

The etching controller may serve to control an etching rate or the like through an interaction with various metal atoms included in the metal-containing film which is a target film to be treated. In addition, the etching controller may serve to remove residues generated during a deposition process and/or a patterning process of the metal-containing film.

The etching controller may include a compound represented by Formula 1:

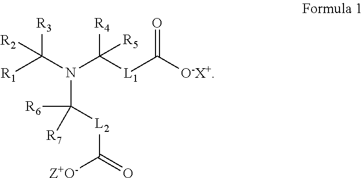

In Formula 1,
R₁ may be a C₁-C₃₀ alkyl group or a C₂-C₃₀ alkenyl group,
R₂ to R₇ are each independently hydrogen, a C₁-C₃₀ alkyl group, or a C₂-C₃₀ alkenyl group,
L₁ and L₂ may each independently be a C₁-C₃₀ alkylene group or a C₂-C₃₀ alkenylene group,
X and Z may each independently be hydrogen, an alkali metal, or an ammonium group,
at least one methylene group included in R₁ to R₇ may optionally be substituted with O or S, and
at least one hydrogen included in R₁ to R₇, L₁, and L₂ may optionally be substituted with a halogen atom, a hydroxyl group, a thiol group, a C₁-C₃₀ alkoxy group, or a C₁-C₃₀ alkylthio group.

In an aqueous solution, two groups each represented by *—C(═O)O⁻ in Formula 1 are capable of strong bonding with metal M (e.g., Co, Cu, etc.) included in a metal-containing film 2 (see two “2a”s in Formula 1′), so that the compound represented by Formula 1 may be effectively fixed on the surface of the metal-containing film 2. Furthermore, a monoamine-based moiety (see “2b” in Formula 1′) including R₁ to R₇, L₁, and L₂ as defined above may have a relatively bulky structure, as all three groups bonded to N included in the moiety have “two or more carbon atoms”. Accordingly, the compound represented by Formula 1 may provide a protective film with excellent surface characteristics on the surface of the metal-containing film 2, since intermolecular interactions are substantially inhibited by the compound represented by Formula 1. Therefore, by using the composition including the compound represented by Formula 1, the etching rate may optionally be adjusted according to the metal included in the metal-containing film, and at the same time, residues generated during a deposition process and/or a patterning process of the metal-containing film may be more effectively removed.

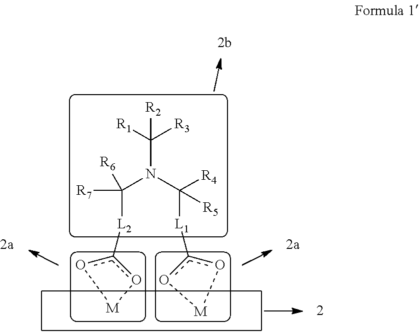

In addition, the compound represented by Formula 1 is substantially uniformly soluble in the composition while substantially not forming micelles, so that bubble formation and phase separation may be substantially limited and/or prevented when preparing the composition and/or treating the metal-containing film by using the composition. The bubble formation and phase separation in the composition may cause a decrease in efficiency and stability of a treatment process on the metal-containing film, damage to wafer, regeneration of residues, contamination of various equipment, and the like, and thus it therefore may be advantageous to limit and/or prevent bubble formation and phase separation during the preparation of the composition or during the treatment of the metal-containing film by using the composition. Although not particularly limited to a specific theory, the composition in which bubble formation and phase separation are observed during the preparation and/or immediately after the preparation may accordingly interfere uniform contact between the metal-containing film and other active ingredients included in the composition and generate another residue on the surface of the metal-containing film, and thus the composition in which bubble formation and phase separation are observed may be substantially unsuitable for use in the treatment of the metal-containing film. Therefore, by using the composition including the compound represented by Formula 1, the etching rate may be optionally adjusted depending on the metal included in the metal-containing film without causing bubble formation and phase separation, and at the same time, residues generated during a deposition process and/or a patterning process of the metal-containing film may be more effectively removed.

In an embodiment, R₁ in Formula 1 may be:
1) a C₃-C₃₀ alkyl group or a C₃-C₃₀ alkenyl group,
2) a C₅-C₃₀ alkyl group or a C₅-C₃₀ alkenyl group,
3) a C₇-C₃₀ alkyl group or a C₇-C₃₀ alkenyl group,
4) a C₃-C₂₀ alkyl group or a C₃-C₂₀ alkenyl group,
5) a C₅-C₂₀ alkyl group or a C₅-C₂₀ alkenyl group,
6) a C₇-C₂₀ alkyl group or a C₇-C₂₀ alkenyl group,
7) a C₃-C₁₁ alkyl group or a C₃-C₁₁ alkenyl group,
8) a C₅-C₁₁ alkyl group or a C₅-C₁₁ alkenyl group, or
9) a C₇-C₁₁ alkyl group or a C₇-C₁₁ alkenyl group.

In one or more embodiments, atoms included in R₁ of Formula 1 may be carbon and hydrogen. When atoms included in R₁ of Formula 1 include carbon and hydrogen (e.g., when R₁ is an unsubstituted C₁-C₃₀ alkyl group or an unsubstituted C₂-C₃₀ alkenyl group), a protective film provided on the surface of the metal-containing film 2 may have improved hydrophobicity due to the compound represented by Formula 1, so that side reactions (e.g., surface oxidation of the metal-containing film) of the metal-containing film in contact with the composition may be limited and/or substantially prevented.

In one or more embodiments, R₂ to R₇ in Formula 1 may each independently be i) hydrogen, a C₁-C₂₀ alkyl group, or a C₂-C₂₀ alkenyl group,
ii) hydrogen, a C₁-C₁₀ alkyl group, or a C₂-C₁₀ alkenyl group,
iii) hydrogen, a C₁-C₅ alkyl group, or a C₂-C₅ alkenyl group,
iv) hydrogen or a methyl group, or
v) hydrogen.

In one or more embodiments, L₁ and L₂ in Formula 1 may each independently be
i) a C₁-C₁₀ alkylene group,
ii) a C₁-C₄ alkylene group,
iii) a C₁-C₂ alkylene group, or
iv) a C₁ alkylene group (a methylene group).

In one or more embodiments, atoms included in each of L₁ and L₂ of Formula 1 may be carbon and hydrogen. When atoms included in each of L₁ and L₂ of Formula 1 are carbon and hydrogen (e.g., each of L₁ and L₂ is an unsubstituted C₁-C₃₀ alkylene group or an unsubstituted C₂-C₃₀ alkenylene group), a protective film provided on the surface of the metal-containing film 2 may have improved hydrophobicity due to the compound represented by Formula 1, so that side reactions (e.g., surface oxidation of the metal-containing film) of the metal-containing film in contact with the composition may be limited and/or substantially prevented.

In one or more embodiments, X and Z in Formula 1 may each independently be hydrogen, Na, K, or N(A₁)(A₂)(A₃)(A₄), wherein A₁ to A₄ may each independently be hydrogen, a C₁-C₃₀ alkyl group, a C₂-C₃₀ alkenyl group, a C₃-C₃₀ carbocyclic group, or a C₁-C₃₀ heterocyclic group. For example, A₁ to A₄ may each independently be hydrogen or a C₁-C₁₀ alkyl group.

In one or more embodiments, at least one methylene group (e.g., one or two methylene groups) included in R₁ to R₇ of Formula 1 may optionally be substituted with O or S.

In one or more embodiments, at least one hydrogen included in R₁ to R₇, L₁, and L₂ of Formula 1 may optionally be substituted with a halogen atom (e.g., —F, —Cl, —Br, etc.), a hydroxyl group, a thiol group, a C₁-C₃₀ alkoxy group (e.g., a C₁-C₁₀ alkoxy group), or a C₁-C₃₀ alkylthio group (e.g., a C₁-C₁₀ alkylthio group).

In one or more embodiments, the etching controller may include at least one of Compounds 1 and 2.

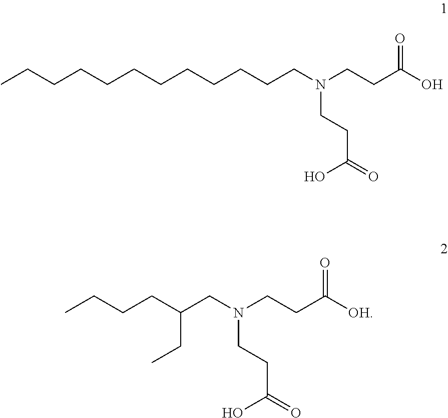 [SMILES: CCCCCCCCCCCCN(CCC(=O)O)CCC(=O)O ; CCCCC(CC)CN(CCC(=O)O)CCC(=O)O]

In one or more embodiments, an amount of the etching controller may be, per 100 wt % of the composition, in a range of about 0.001 wt % to about 10 wt %, about 0.01 wt % to about 10 wt %, about 0.1 wt % to about 10 wt %, about 0.2 wt % to about 10 wt %, about 0.001 wt % to about 5 wt %, about 0.01 wt % to about 5 wt %, about 0.1 wt % to about 5 wt %, about 0.2 wt % to about 5 wt %, about 0.001 wt % to about 1 wt %, about 0.01 wt % to about 1 wt %, about 0.1 wt % to about 1 wt %, about 0.2 wt % to about 1 wt %, about 0.001 wt % to about 0.5 wt %, about 0.01 wt % to about 0.5 wt %, about 0.1 wt % to about 0.5 wt %, about 0.2 wt % to about 0.5 wt %, about 0.001 wt % to about 0.2 wt %, about 0.01 wt % to about 0.2 wt %, or about 0.1 wt % to about 0.2 wt %.

A pH of the composition may be in a range of about 1.0 to about 12.0, about 3.0 to about 12.0, about 5.0 to about 12.0, about 7.0 to about 12.0, about 1.0 to about 10.0, about 3.0 to about 10.0, about 5.0 to about 10.0, about 7.0 to about 10.0, about 3.0 to about 8.0, about 5.0 to about 8.0, or about 7.0 to about 8.0. When the pH of the composition is within the ranges above, interactions between the etching controller and metal atoms included in the metal-containing film may be more smoothly achieved.

In an embodiment, the composition may be used in a process of treating the metal-containing film, such as an etching process, a cleaning process, etc., on the metal-containing film. The metal-containing film is the same as described elsewhere herein.

In an embodiment, the composition may be used as a scavenger of an etching by-product, a scavenger of a post-etch process by-product, a scavenger of an ashing process by-product, a cleaning composition, a photoresist (PR) scavenger, an etching composition for packaging process, a cleaning agent for packaging process, a removing agent for adhesive substances of wafer, an etchant, a post-etch residue stripper, an ash residue cleaner, a PR residue stripper, a post-CMP cleaner, or the like.

### Method of Treating Metal-Containing Film and Method of Manufacturing Electronic Device

By using the aforementioned composition, the metal-containing film may be more effectively treated.

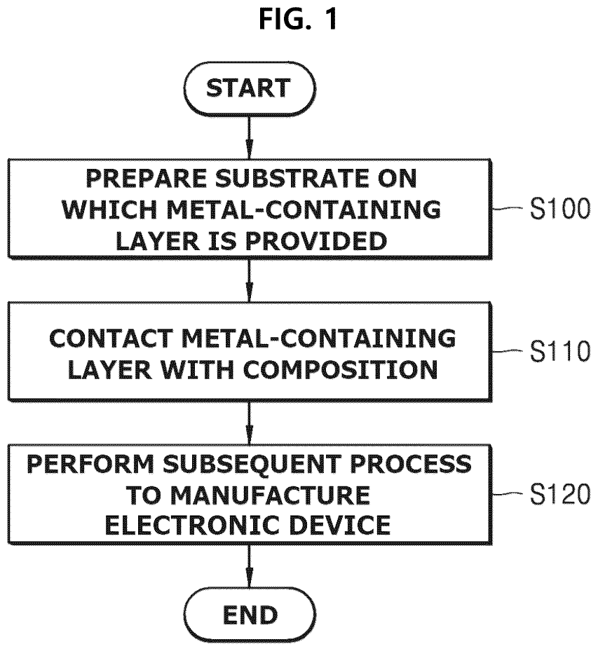

Referring to FIG. 1, a method of treating the metal-containing film according to an embodiment may include: preparing a substrate on which a metal-containing film is provided (S100); and contacting the metal-containing film with the composition (S110).

The metal-containing film is the same as described elsewhere herein.

For example, a metal included in the metal-containing film may include Ti, In, Al, Co, La, Sc, Ga, W, Mo, Ru, Zn, Hf, Cu, or any combination thereof.

In one or more embodiments, the metal-containing film may include a metal, metal nitride, metal oxide, metal oxynitride, or any combination thereof.

In one or more embodiments, the metal-containing film may include a metal, metal nitride, metal oxide, metal oxynitride, or any combination thereof, and each of the aforementioned metal, a metal included in the metal nitride, a metal included in the metal oxide, and a metal included in the metal oxynitride may include Ti, In, Al, Co, La, Sc, Ga, W, Mo, Ru, Zn, Hf, Cu, or any combination thereof.

In one or more embodiments, the metal-containing film may include titanium nitride.

In one or more embodiments, the metal-containing film may include Co, Cu, or a combination thereof.

In an embodiment, by the contacting of the metal-containing film with the composition, at least a portion of the metal-containing film may be etched and/or cleaned.

In the composition, i) the oxidizing agent may serve to etch at least a portion of the metal-containing film by oxidizing at least some of metals included in the metal-containing film and forming a water-soluble complex, ii) the ammonium-based buffer may serve to maintain a high concentration of anions generated by the oxidizing agent and to stabilize a water-soluble complex generated when the anions oxidize at least a portion of a metal included in the metal-containing film, thereby effectively etching at least a portion of the metal-containing film, iii) the compound represented by Formula 1 included in the etching controller has two groups each represented by *—C(═O)O⁻ being capable of strong bonding with a metal (e.g., Co, Cu, etc.) included in the metal-containing film and the monoamine-based moiety including R₁ to R₇, L₁, and L₂ as defined above which may substantially inhibit intermolecular interactions so that a protective film having excellent surface characteristics and additionally improved hydrophobicity may be provided on the surface of the metal-containing film. Thus, by using the etching controller including the compound represented by Formula 1, the etching rate may selectively be adjusted depending on metals included in the metal-containing film, and at the same time, residues generated during a deposition process and/or a patterning process of the metal-containing film may be effectively removed. Therefore, the aforementioned composition may be utilized in various treatment processes for the metal-containing film.

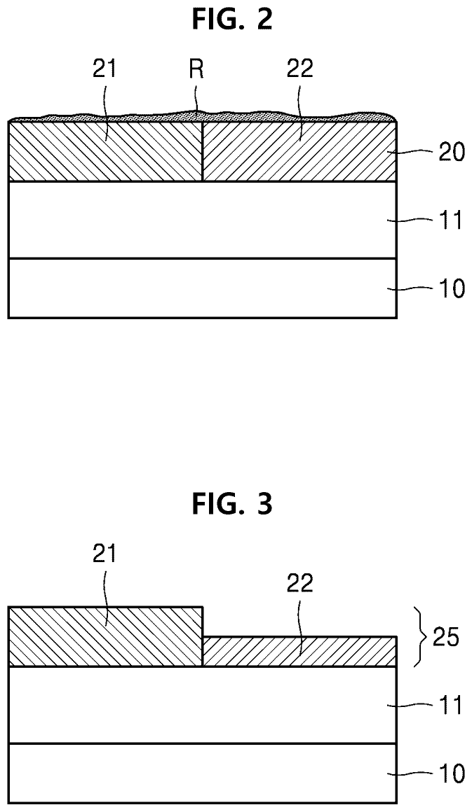

FIGS. 2 and 3 are each a diagram briefly explaining an embodiment of a method of treating a metal-containing film.

Referring to FIG. 2, a substrate 10 on which a metal-containing film 20 is provided may be provided. An interlayer 11 may be arranged between the substrate 10 and the metal-containing film 20. Although not shown in FIG. 2, circuitry elements (e.g., a transistor gate, metal lines, impurity regions, semiconductor layers, or the like) may be arranged inside the substrate 10, on the substrate 10, and/or between the substrate 10 and the interlayer 11. In an embodiment, the metal-containing film 20 may be arranged directly on the substrate 10, and the interlayer 11 may be omitted.

The metal-containing film 20 may include a first region 21 and a second region 22. The first region 21 and the second region 22 may be spaced apart from each other, or at least a portion of the first region 21 and at least a portion of the second region 22 may be in direct contact with each other, and in this regard, the metal-containing film 20 may have various three-dimensional patterns. A second etching rate of the composition for etching the second region 22 may be greater than a first etching rate of the composition for etching the first region 21. For example, when the first etching rate is 0, the first region may not be etched.

Referring to FIG. 3, the composition may be used to etch the metal-containing film 20 so that at least a portion of the second region 22 may be etched, thereby forming a metal-containing film pattern 25. The etching process may be performed by contacting the composition with at least a portion of the first region 21 with at least a portion of the second region 22.

In an embodiment, the composition may be used to etch only at least a portion of the second region 22 without etching the first region 21. In one or more embodiments, the composition may be used to etch each of at least a portion of the first region 21 and at least a portion of the second region 22. Referring to FIG. 3, the metal-containing film pattern 25 formed after etching may include at least a portion of the second region 22, but, if desired, the etching process may be performed to completely remove the second region 22 from the metal-containing film pattern 25. In this regard, various modifications may be possible.

In one or more embodiments, the first region 21 may include Co, Cu, or a combination thereof.

In one or more embodiments, the second region 22 may include metal nitride (e.g., titanium nitride).

In one or more embodiments, the second region 22 may include i) titanium nitride, ii) titanium nitride further including In, Al, La, Sc, Ga, Zn, Hf, or any combination thereof (e.g., TiAlN), or iii) a combination thereof.

In one or more embodiments, each of the first region 21 and the second region 22 may include i) titanium nitride, ii) titanium nitride further including In, Al, La, Sc, Ga, Zn, Hf, or any combination thereof, or iii) a combination thereof.

In one or more embodiment, the first region 21 may include Co, Cu, or a combination thereof, whereas the second region 22 may not include Co and Cu.

In one or more embodiments, the first region 21 may include Co, Cu, or a combination thereof, and the second region 22 may include i) titanium nitride, ii) titanium nitride further including In, Al, La, Sc, Ga, Zn, Hf, or any combination thereof (e.g., TiAlN), or iii) a combination thereof.

In one or more embodiments, the first region 21 may include Co, Cu, or a combination thereof, and the second region 22 may include titanium nitride, titanium nitride further including Al (e.g., TiAlN), or any combination thereof.

In one or more embodiments, the first region 21 may include a Co film, a Cu film, or a combination thereof, and the second region 22 may include a titanium nitride film, a titanium nitride film further including Al (e.g., a titanium/aluminum nitride film or a TiAlN film), or any combination thereof.

In one or more embodiments, the first region 21 may be a Co film, and the second region 22 may be a titanium nitride film or a titanium nitride film further including Al (e.g., TiAlN film).

In one or more embodiments, the first region 21 may be a Cu film, and the second region 22 may be a titanium nitride film or a titanium nitride film further including Al (e.g., TiAlN film).

In one or more embodiments, by the contacting of the metal-containing film 20 with the composition, residues R on the surface of the metal-containing film 20 may be removed so that at least a portion of the metal-containing film 20 may be cleaned. Thus, as shown in FIG. 3, the metal-containing film pattern 25 that is free of the residues R may be formed.

The residues R are by-products produced during deposition and/or patterning of the metal-containing film 20, and may be substances that remain on the surface of the metal-containing film 20 and/or on the surface of the metal-containing film pattern 25 and accordingly cause an increase in electrical resistance and/or an electrical short between electrical wiring. The residues R may be etching residues produced as a result of etching, and may include, for example, etching gas residues, polymer residues, metal-containing residues, or any combination thereof.

The etching gas residue may be derived from etching gas used for dry etching. The etching gas may be, for example, fluorocarbon gas. For example, the etching gas may include CHF₃, C₂F₆, CF₄, C₄F₈, C₂HF₅, and the like. The etching gas residue may include the etching gas itself and/or a reaction product from any substance in contact with the etching gas during an etching process using the etching gas.

The polymer residues may be polymers derived from various organic substances included in a photoresist, a dielectric layer, a buffer layer, a diffusion barrier layer, etc. that are used during manufacture and/or patterning of the metal-containing film 20. For example, the polymer residues may be polymers including C, Si, F, or any combination thereof.

The metal-containing residues may be any residue including a metal separated from the metal-containing film 20 during manufacture and/or patterning of the metal-containing film 20.

Referring to FIG. 1, a method of manufacturing an electronic device according to an embodiment may include: preparing a substrate on which a metal-containing film is provided (S100); contacting the metal-containing film with the composition (S110); and manufacturing an electronic device by performing one or more subsequent manufacturing process(es) (S120).

In an embodiment, the electronic device may be a semiconductor device.

For example, the preparing of a substrate on which a metal-containing film is provided (S100) and the contacting of the metal-containing film with the composition (S110) may be used in a trench-via hole patterning process for bitline electrode formation as a part of a method of manufacturing an electronic device such as a semiconductor device.

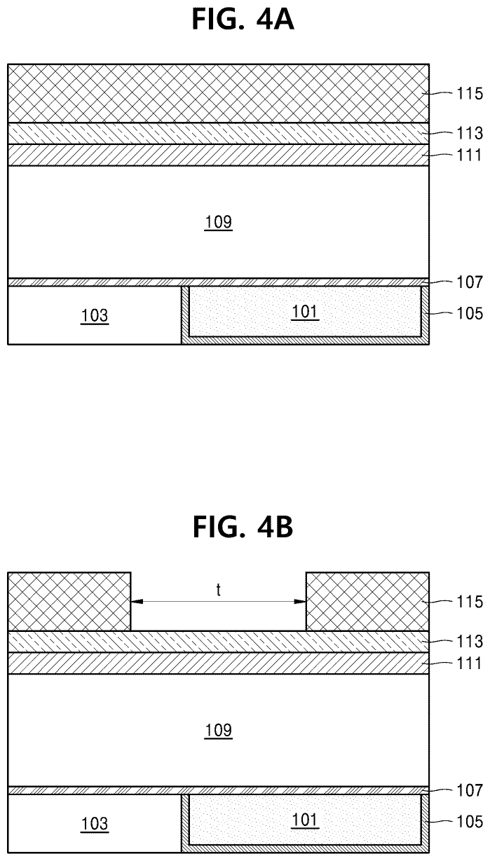

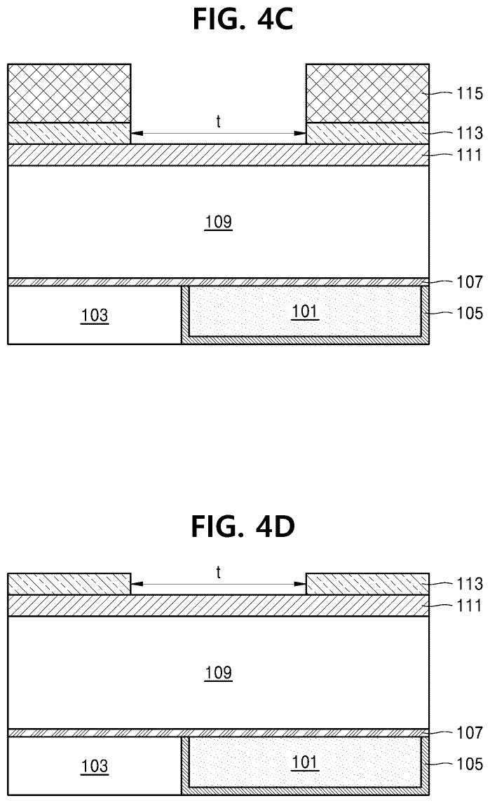

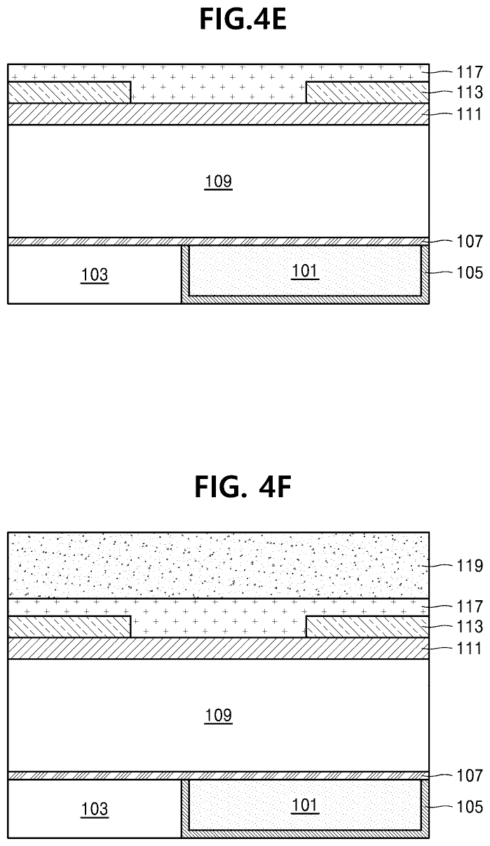

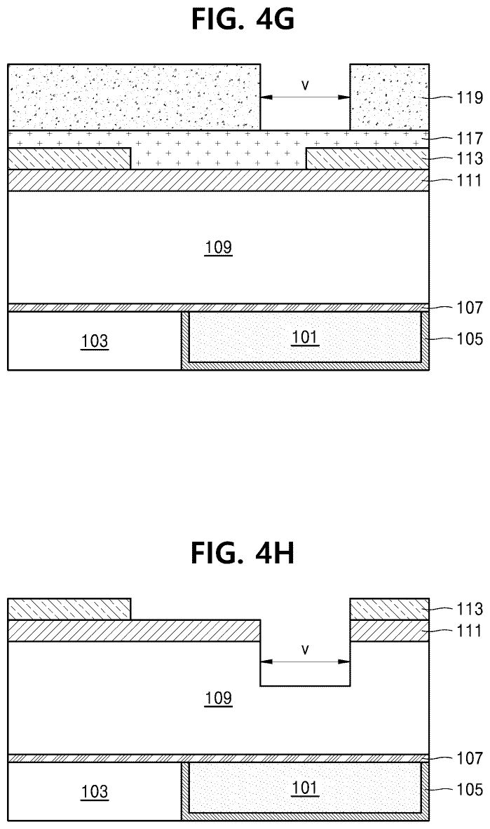

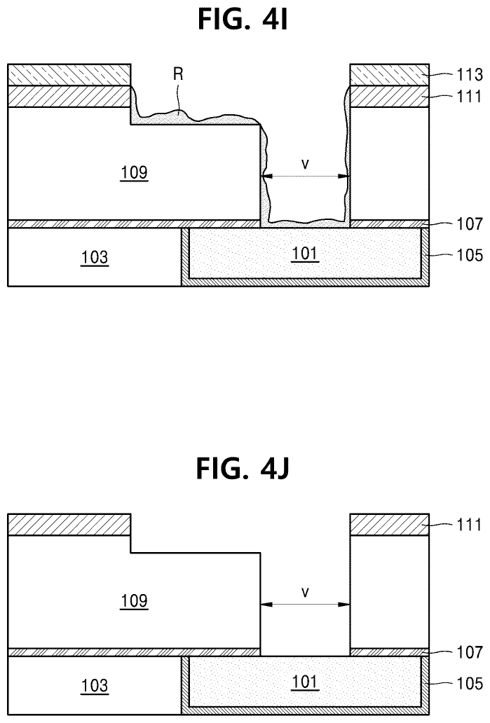

Hereinafter, referring to FIGS. 4A to 4J, an embodiment of using the composition for a trench-via hole patterning process for bitline electrode formation is described.

FIG. 4A depicts a portion of a semiconductor substrate (in which a transistor and the like is not shown) including a first dielectric layer 103 and a metal layer 101. The metal layer 101 may include, for example, Co, Cu, or a combination thereof. A first diffusion barrier layer 105 may be arranged between the first dielectric layer 103 and the metal layer 101. The first diffusion barrier layer 105 may include, for example, Ta, Ti, W, tantalum nitride, titanium nitride, tungsten nitride, or any combination thereof.

A second diffusion barrier layer 107 may be arranged over the first dielectric layer 103 and the metal layer 101 of FIG. 4A. The second diffusion barrier layer 107 may include, for example, silicon nitride, nitrogen-doped silicon carbide, or aluminum oxide.

A second dielectric layer 109 may be arranged on the second diffusion barrier layer 107 of FIG. 4A. The second dielectric layer 109 may include, for example, an ultra-low K (ULK) dielectric or silicon oxide.

On the second dielectric layer 109 of FIG. 4A, a mechanically robust buffer layer 111 may be arranged to limit and/or prevent damage to the second dielectric layer 109 during deposition of a hard mask layer 113. The buffer layer 111 may include, for example, tetraethyl orthosilicate (TEOS), carbon-doped silicon oxide (SiCOH), or the like.

A hard mask layer 113 may be arranged on the buffer layer 111 of FIG. 4A. The hard mask layer 113 may include i) titanium nitride, ii) titanium nitride further including In, Al, La, Sc, Ga, Zn, Hf, or any combination thereof (e.g., TiAlN), or iii) a combination thereof. For example, the hard mask layer 113 may include titanium nitride.

A first photoresist 115 may be arranged on the hard mask layer 113 of FIG. 4A.

Subsequently, by patterning the first photoresist 115, a pattern of the first photoresist 115 having a first opening with a width t may be formed as shown in FIG. 4B, and by etching the hard mask layer 113 according to the pattern of the first photoresist 115, a pattern of the hard mask layer 113 may be formed and a portion of the buffer layer 111 may be opened as shown in FIG. 4C. For example, by ashing, the pattern of the first photoresist 115 may be removed to form an exposed pattern of the hard mask layer 113 as shown in FIG. 4D.

Subsequently, as shown in FIG. 4E, a filler layer 117 may be formed to cover the pattern of the hard mask layer 113 so that the opening in the pattern of the mask layer 113 may be filled. The filler layer 117 may include, for example, hydrogen silsesquioxane (HSQ), methyl silsesquioxane (MSQ), or the like.

Next, after forming a second photoresist 119 on the filler layer 117 as shown in FIG. 4F, the second photoresist 119 may be patterned to form a pattern of the second photoresist 119 having a second opening with a width V as shown in FIG. 4G. For example, by reactive ion etching (RIE) or the like, the filler layer 117, a portion of the pattern of the hard mask layer 113, a portion of the buffer layer 111, and a portion of the second dielectric layer 109 that are positioned below the pattern of the second photoresist 119 may be etched to partially form a via hole as shown in FIG. 4H. Then, the pattern of the second photoresist 119 and the filler layer 117 may be removed.

Next, as shown in FIG. 4I, the buffer layer 111, the second dielectric layer 109, and the second diffusion barrier layer 107 may be etched by, for example, a dry etching process, according to the pattern of the hard mask layer 113 until the via hole reaches the metal layer 101, thereby forming a trench-via hole pattern. Etching gas used in the dry etching process may be, for example, fluorocarbon gas (e.g., CHF₃, C₂F₆, CF₄, C₄F₈, C₂HF₅, etc.).

As a result of the dry etching, a large amount of residues R may exist on the inner wall of the trench-via hole pattern as shown in FIG. 4I. The residues R may include etching gas residues, polymer residues, metal-containing residues, or any combination thereof. The etching gas residues may include the etching gas itself and/or a product resulting from a reaction with any substance (e.g., a material included in the buffer layer 111, the second dielectric layer 109, and the like) that is in contact with etching gas during the etching process using the etching gas. The polymer residues may include polymers derived from various organic materials included in the second photoresist 119, the second dielectric layer 109, the buffer layer 111, the second diffusion barrier layer 107, and the like. For example, the polymer residues may be polymers including C, Si, F, or any combination thereof. The metal-containing residues may include, for example, residues including metals in the pattern of the hard mask layer 113.

The residues R shown in FIG. 4I may increase the electrical resistance of a semiconductor device or cause an electrical short circuit of a bitline electrode which will be formed later, and thus it may be advantageous to remove the residues R. Meanwhile, to simplify the process, it may be advantageous to remove the residues R and the pattern of the hard mask layer 113 at the same time. In addition, during the removal of the residues R and the removal of the pattern of the hard mask layer 113, the metal layer 101 is not substantially removed.

For this purpose, when the composition including the oxidizing agent, the ammonium-based buffer, and the etching controller is in contact with the substrate of FIG. 4I including the metal-containing film that includes the pattern of the hard mask layer 113 and the metal layer 101, a substrate of FIG. 4J, in which i) the residues R produced on the inner walls of the trench-via hole pattern may be removed, ii) the pattern of the hard mask layer 113 may be removed, and iii) the metal layer 101 is substantially undamaged, may be prepared. Although not particularly limited to a specific theory, for example, the pattern of the hard mask layer 113 may be removed by the oxidizing agent and the ammonium-based buffer, the residues R may be removed by the etching controller, and at the same time, the metal layer 101 may not be substantially etched. Afterwards, a bitline electrode or the like may be formed by filling the trench-via hole pattern with a metallic material or the like.

### Examples 1 and 2 and Comparative Examples R1 to R4

25 wt % of hydrogen peroxide, 0.5 wt % of (NH₄)₂HPO₄, 0.5 wt % of tetramethyl ammonium hydroxide (TMAH), 0.1 wt % of ethylenediaminetetraacetic acid (EDTA), and a compound shown in Table 1 as an etching controller at an amount shown in Table 1 were weighed and mixed, to prepare compositions of Examples 1 and 2 and Comparative Examples R1 to R4. The remainders of each composition correspond to water (deionized water).

### Comparative Example R5

25 wt % of hydrogen peroxide, 0.5 wt % of (NH₄)₂HPO₄, 0.5 wt % of TMAH, and 0.1 wt % of EDTA were mixed to prepare a composition of Comparative Example R₅. The remainders of the composition correspond to water (deionized water).

### Evaluation Example 1

Regarding the composition of Example 1, a PH meter was used to evaluate the pH, and whether bubbles were formed or phases were separated in the composition was evaluated with naked eyes, and the results are summarized in Table 1.

Next, a substrate having a trench-via hole pattern formed for the formation of a bitline electrode, and having residues present on the inner wall of the trench-via hole pattern was immersed in a cleaning bath (a dip-type bath) containing the composition of Example 1 (at 25° C.). After performing a rinse and drying process on the substrate, whether the residues were removed was evaluated by atomic force microscopy (AFM) topography analysis, and the results are summarized in Table 1. The substrate is the one having a trench-via hole pattern as shown in FIG. 4I in which the metal layer 101 includes copper, the second dielectric layer 109 includes silicon oxide, the hard mask layer 113 includes titanium nitride, the buffer layer 111 includes carbon-doped silicon oxide, the second diffusion barrier layer 107 includes aluminum oxide, and the etching gas used in the dray etching process is CF₄.

The same test was repeated by using each of the compositions of Example 2 and Comparative Examples R1 to R5, and the results are summarized in Table 1.

TABLE 1

| | Etching | | | | | |
|---|---|---|---|---|---|---|
| | controller | | | | | |
| | Com- | Amount | | Bubble | Phase | Removal of |
| Division | pound | (wt %) | pH | formation | separation | residues |
| Example 1 | 1 | 0.2 | 7.5 | N | N | Good |
| Example 2 | 2 | 0.2 | 7.5 | N | N | Good |
| Comparative | C | 0.2 | 7.5 | N | N | Poor |
| Example R1 | | | | | | |
| Comparative | D | 0.2 | 7.5 | Y | Y | Poor |
| Example R2 | | | | | | |
| Comparative | E | 0.2 | 7.5 | Y | N | Poor |
| Example R3 | | | | | | |
| Comparative | F | 0.2 | 7.5 | N | N | Poor |
| Example R4 | | | | | | |
| Comparative | — | — | 7.5 | N | N | Poor |
| Example R5 | | | | | | |

Bubble formation “N”: Bubble formation not observed in composition
Bubble formation “Y”: Bubble formation observed in composition
Phase separation “N”: Phase separation not observed in composition
Phase separation “Y”: Phase separation observed in composition
Good: No residues with a length of 10 nm or more observed
Poor: Residues with a length of 10 nm or more observed

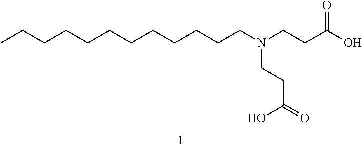 [SMILES: CCCCCCCCCCCCN(CCC(=O)O)CCC(=O)O]

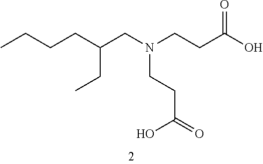 [SMILES: CCCCC(CC)CN(CCC(=O)O)CCC(=O)O]

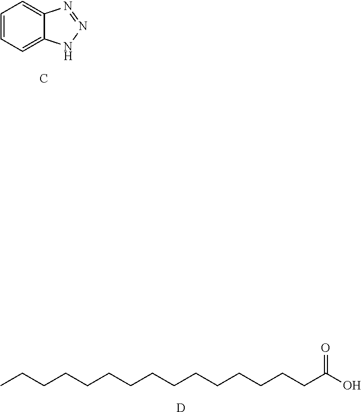

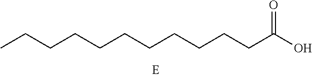

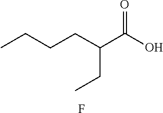

Referring to Table 1, it was confirmed that the compositions of Examples 1 and 2 had improved and/or excellent residual removal performance compared to the compositions of Comparative Examples R1 to R5. In addition, it was confirmed that the compositions of Examples 1 and 2 had improved and/or excellent stability since bubble formation and phase separation were not observed, unlike the compositions of Comparative Examples R2 and R3.

### Evaluation Example 2

The composition of Example 1 was added to two beakers and heated to 50° C., and a Cu film and a titanium nitride film were immersed in each beaker for 10 minutes and 0.5 minutes, respectively. Then, a thickness of the Cu film was measured with an X-ray fluorescence (XRF) spectrometry meter (S8 Tiger, BRUKER), and a thickness of the titanium nitride film was measured with an ellipsometer (M-2000, J. A. Woolam). Accordingly, an etching rate (Å/min) of the composition to the Cu film and an etching rate (Å/min) of the composition to the titanium nitride film were evaluated, and the etching rate for the titanium nitride film was divided by the etching rate for the Cu film to evaluate R(TiN/Cu), and the results are summarized in Table 2.

The same test was repeated by using each of the compositions of Example 2 and Comparative Examples R1 and R5, and the results are summarized in Table 2.

TABLE 2

| | Etching | | Etching | Etching rate | |
|---|---|---|---|---|---|
| | controller | | rate for | for titanium | |
| | | Amount | Cu film | nitride film | |
| Division | Compound | (wt %) | (Å/min) | (Å/min) | R(TiN/Cu) |
| Example 1 | 1 | 0.2 | 0.8 | 176.5 | 220.6 |
| Example 2 | 2 | 0.2 | 1.0 | 171.5 | 171.5 |
| Comparative | C | 0.2 | 2.4 | 172.3 | 71.8 |
| Example R1 | | | | | |
| Comparative | — | — | 1.4 | 174.4 | 124.6 |
| Example R5 | | | | | |

Referring to Table 2, it was confirmed that the compositions of Examples 1 and 2 had improved and/or excellent etching inhibition performance on a copper film and improved and/or excellent etching selectivity of a titanium nitride film to a copper film, compared to the compositions of Comparative Examples R1 and R5.

### Evaluation Example 3

The composition of Example 1 was added to two beakers and heated to 50° C., and a Co film and a titanium nitride film were immersed in each beaker for 5 minutes and 0.5 minutes, respectively. Then, a thickness of the Co film was measured with an XRF spectrometry meter (S8 Tiger, BRUKER), and a thickness of the titanium nitride film was measured with an ellipsometer (M-2000, J. A. Woolam). Accordingly, an etching rate (Å/min) of the composition to the Co film and an etching rate (Å/min) of the composition to the titanium nitride film were evaluated, and the etching rate for the titanium nitride film was divided by the etching rate for the Co film to evaluate R(TiN/Co), and the results are summarized in Table 3.

The same test was repeated by using each of the compositions of Example 2 and Comparative Examples R4 and R5, and the results are summarized in Table 3.

TABLE 3

| | | | | Etching | |
|---|---|---|---|---|---|
| | Etching | | Etching | rate for | |
| | controller | | rate for | titanium | |
| | | Amount | Co film | nitride film | |
| Division | Compound | (wt %) | (Å/min) | (Å/min) | R(TiN/Co) |
| Example 1 | 1 | 0.2 | 1.0 | 176.5 | 176.5 |
| Example 2 | 2 | 0.2 | 4.7 | 171.5 | 36.5 |
| Comparative | F | 0.2 | 10.9 | 161.8 | 14.8 |
| Example R4 | | | | | |
| Comparative | — | — | >10 | 174.4 | — |
| Example R5 | | | | | |

Referring to Table 3, it was confirmed that the compositions of Examples 1 and 2 had improved and/or excellent etching inhibition performance on a cobalt film and improved and/or excellent etching selectivity of a titanium nitride film to a cobalt film, compared to the compositions of Comparative Examples R4 and R5.

### Comparative Examples R11 to R13

25 wt % of hydrogen peroxide, 0.5 wt % of (NH₄)₂HPO₄, 0.5 wt % of TMAH, 0.1 wt % of EDTA, and a compound shown in Table 4 as an etching controller at an amount shown in Table 4 were mixed, to prepare compositions of Comparative Examples R11 to R13. The remainders of each composition correspond to water (deionized water).

### Evaluation Example 4

Regarding Comparative Example R11 to R13, the pH, etching rate (Å/min) for a Cu film, etching rate (Å/min) for a Co film, etching rate (Å/min) for a titanium nitride film, R(TiN/Cu), and R(TiN/Co) were each evaluated in the same manner as in Evaluation Examples 2 and 3, and the results are summarized in Table 4 together with data of Examples 1 and 2.

TABLE 4

| | | | | | | Etching | | |
|---|---|---|---|---|---|---|---|---|
| | | | | | | rate for | | |
| | Etching | | | Etching | Etching | titanium | | |
| | controller | | | rate for | rate for | nitride | | |
| | Com- | Amount | | Cu film | Co film | film | R | R |
| Division | pound | (wt %) | pH | (Å/min) | (Å/min) | (Å/min) | (TiN/Cu) | (TiN/Co) |
| Example 1 | 1 | 0.2 | 7.5 | 0.8 | 1.0 | 176.5 | 220.6 | 176.5 |
| Example 2 | 2 | 0.2 | 7.5 | 1.0 | 4.7 | 171.5 | 171.5 | 36.5 |
| Comparative | H | 0.2 | 7.5 | 1.5 | >10 | 167.8 | 111.9 | — |
| Example R11 | | | | | | | | |
| Comparative | I | 0.2 | 7.5 | 1.5 | >10 | 164.3 | 109.5 | — |
| Example R12 | | | | | | | | |
| Comparative | K | 0.2 | 7.5 | 1.9 | >10 | 170.3 | 89.6 | — |
| Example R13 | | | | | | | | |

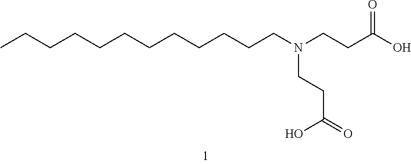 [SMILES: CCCCCCCCCCCCN(CCC(=O)O)CCC(=O)O]

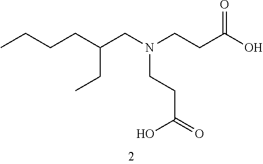 [SMILES: CCCCC(CC)CN(CCC(=O)O)CCC(=O)O]

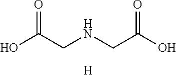

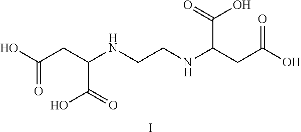

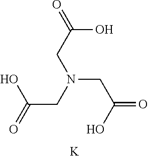

Referring to Table 4, it was confirmed that the compositions of Examples 1 and 2 had improved and/or excellent characteristics, in terms of etching inhibition performance on a Cu film, etching selectivity of a titanium nitride film to a copper film, etching inhibition performance on a cobalt film, and etching selectivity of a titanium nitride film to a copper film, compared to the compositions of Comparative Examples R11 to R13.

According to the one or more embodiments, a composition has improved etching selectivity and improved cleaning performance, and thus may be more effectively used in various treatment processes for a metal-containing film, such as etching, cleaning, etc. In this regard, when a metal-containing film is treated by using the composition, a higher-quality electronic device, e.g., semiconductor device, may be manufactured.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A composition comprising
an oxidizing agent;
an ammonium-based buffer; and
an etching controller,
wherein the etching controller includes a compound represented by Formula 1, 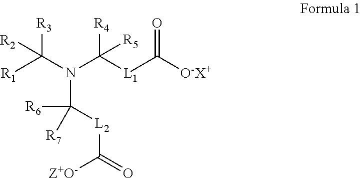
wherein, in Formula 1,
R₁ is a C₁-C₃₀ alkyl group or a C₂-C₃₀ alkenyl group,
R₂ to R₇ are each independently hydrogen, a C₁-C₃₀ alkyl group, or a C₂-C₃₀ alkenyl group,
L₁ and L₂ are each independently a C₁-C₃₀ alkylene group or a C₂-C₃₀ alkenylene group,
X and Z are each independently hydrogen, an alkali metal, or an ammonium group,
at least one methylene group included in one or more of R₁ to R₇ is optionally substituted with O or S, and
at least one hydrogen included in one or more of R₁ to R₇, L₁, and L₂ is optionally substituted with a halogen atom, a hydroxyl group, a thiol group, a C₁-C₃₀ alkoxy group, or a C₁-C₃₀ alkylthio group.

2. The composition of claim 1, wherein the oxidizing agent includes hydrogen peroxide.

3. The composition of claim 1, wherein an amount of the oxidizing agent is in a range of about 16 wt % to about 50 wt % per 100 wt % of the composition.

4. The composition of claim 1, wherein
the ammonium-based buffer includes an ammonium group represented by N(A₁₁)(A₁₂)(A₁₃)(A₁₄), and
A₁₁ to A₁₄ are each independently hydrogen, a C₁-C₃₀ alkyl group, a C₂-C₃₀ alkenyl group, a C₃-C₃₀ carbocyclic group, or a C₁-C₃₀ heterocyclic group.

5. The composition of claim 1, wherein the ammonium-based buffer includes hydroxide, acetate, bicarbonate, benzoate, carbonate, formate, nitrate, hydrogensulfate, carbamate, sulfamate, citrate, phosphate, sulfite, sulfobenzoate, oxalate, lactate, tartrate, dihydrogencitrate, glutamate, salicylate, bioxalate, octanoate, propionate, glycolate, gluconate, or any combination thereof.

6. The composition of claim 1, wherein
the ammonium-based buffer includes a compound represented by Formula 11-1, a compound represented by Formula 11-2, a compound represented by Formula 11-3, a compound represented by Formula 11-4, or any combination thereof: 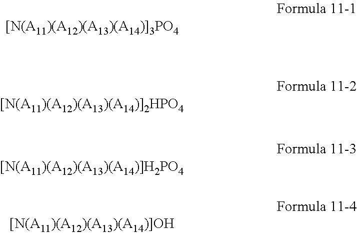
wherein, in Formulae 11-1 to 11-4, A₁₁ to A₁₄ are each independently hydrogen, a C₁-C₃₀ alkyl group, a C₂-C₃₀ alkenyl group, a C₃-C₃₀ carbocyclic group, or a C₁-C₃₀ heterocyclic group.

7. The composition of claim 1, wherein an amount of the ammonium-based buffer is in a range of about 0.01 wt % to about 10 wt % per 100 wt % of the composition.

8. The composition of claim 1, wherein R₁ in Formula 1 is a C₅-C₃₀ alkyl group or a C₅-C₃₀ alkenyl group.

9. The composition of claim 1, wherein atoms included in R₁ in Formula 1 are carbon and hydrogen.

10. The composition of claim 1, wherein L₁ and L₂ in Formula 1 are each independently a C₁-C₄ alkylene group.

11. The composition of claim 1, wherein an amount of the etching controller is in a range of about 0.001 wt % to about 10 wt % per 100 wt % of the composition.

12. The composition of claim 1, wherein the composition has a pH in a range of about 5.0 to about 12.0.

13. A method of treating a metal-containing film, the method comprising:
preparing a substrate on which a metal-containing film is provided; and
contacting the metal-containing film with the composition of claim 1.

14. The method of claim 13, wherein a metal included in the metal-containing film includes titanium (Ti), indium (In), aluminum (Al), cobalt (Co), lanthanum (La), scandium (Sc), gallium (Ga), tungsten (W), molybdenum (Mo), ruthenium (Ru), zinc (Zn), hafnium (Hf), copper (Cu), or any combination thereof.

15. The method of claim 13, wherein the metal-containing film includes a metal, a metal nitride, a metal oxide, a metal oxynitride, or any combination thereof.

16. The method of claim 13, wherein, by the contacting of the metal-containing film with the composition, at least a portion of the metal-containing film is at least one of etched or cleaned.

17. The method of claim 13, wherein
the metal-containing film has a first region and a second region, and
a second etching rate of the composition for etching the second region is greater than a first etching rate of the composition for etching the first region.

18. The method of claim 17, wherein the first region includes Co, Cu, or a combination thereof, and the second region includes titanium nitride.

19. The method of claim 13, wherein
by the contacting of the metal-containing film with the composition, at least a portion of the metal-containing film is cleaned by removing residues on a surface of the metal-containing film, and
the residues include etching gas residues, polymer residues, metal-containing residues, or any combination thereof.

20. A method of manufacturing an electronic device, the method comprising:
preparing a substrate on which a metal-containing film is provided;
contacting the metal-containing film with the composition of claim 1; and
manufacturing the electronic device by performing one or more subsequent manufacturing process(es).
